(19) United States
(12) Patent Application Publication
(10) Pub. No.: US 2005/0282871 A1
(43) Pub. Date: Dec. 22, 2005

(54) 3-(3,5-Disubstituted-4-hydroxyphenyl)propionamide derivatives as cathepsin b inhibitors

(76) Inventors: Leland C. Burrill II, San Francisco, CA (US); James T. Palmer, Madera, CA (US); Robert Rydzewski, Newark, CA (US)

Correspondence Address: CELERA, AN APPLERA CORPORATION BUSINESS, 180 KIMBALL WAY, SOUTH SAN FRANCISCO, CA, 94080, US

(21) Appl. No.: 10/528,266

(22) PCT Filed: Sep. 16, 2003
(86) PCT No.: PCT/US03/29545

§ 371 Date: Mar. 17, 2005

Related U.S. Application Data
(60) Provisional application No. 60/412,368, filed on Sep. 20, 2002.

(51) Int. Cl. C07D 213/57; A61K 31/277; A61K 31/44

(52) U.S. Cl. 514/357; 514/521; 546/330; 558/392

(57) ABSTRACT

The present invention is directed to novel 3-(3,5-disubstituted-4-hydroxyphenyl)-propionamide derivatives that are inhibitors of Cathepsin B. Pharmaceutical composition comprising these compounds, method of treating diseases mediated by Cathepsin B, utilizing these compounds and methods of preparing these compounds are also disclosed.

### BACKGROUND OF THE INVENTION

1. Field of Invention

The present invention is directed to novel 3-(3,5-disubstituted-4-hydroxyphenyl)-propionamide derivatives that are inhibitors of Cathepsin B. Pharmaceutical composition comprising these compounds, method of treating diseases mediated by Cathepsin B utilizing these compounds and methods of preparing these compounds are also disclosed.

2. State of the Art

Cysteine proteases such as Cathepsins B, H, K, L, O and S, represent a class of peptidases characterized by the presence of a cysteine residue in the catalytic site of the enzyme. Cysteine proteases are associated with the normal degradation and processing of proteins. The aberrant activity of cysteine proteases, e.g., as a result of increase expression or enhanced activation, however, may have pathological consequences. In this regard, certain cysteine proteases are associated with a number of disease states, including arthritis, muscular dystrophy, inflammation, tumor invasion, glomerulonephritis, malaria, periodontal disease, metachromatic leukodystrophy and others. For example, increased Cathepsin B levels and redistribution of the enzyme are found in tumors thus suggesting a role for the enzyme in tumor invasion and metastasis. In addition, aberrant Cathepsin B activity is implicated in such disease states as rheumatoid arthritis, osteoarthritis, pneumocystis carinii, acute pancreatitis, inflammatory airway disease and bone and joint disorders.

The prominent expression of Cathepsin K in osteoclasts and osteoclast-related multinucleated cells and its high collagenolytic activity suggest that the enzyme is involved in ososteoclast-mediated bone resorption and, hence, in bone abnormalities such as occurs in osteoporosis. In addition, Cathepsin K expression in the lung and its elastinolytic activity suggest that the enzyme plays a role in pulmonary disorders as well.

Cathepsin L is implicated in normal lysosomal proteolysis as well as several disease states, including, but not limited to, metastasis of melanomas. Cathepsin S is implicated in Alzheimer's disease and certain autoimmune disorders, including, but not limited to juvenile onset diabetes, multiple sclerosis, pemphigus vulgaris, Graves' disease, myasthenia gravis, systemic lupus erythemotasus, rheumatoid arthritis and Hashimoto's thyroiditis. In addition, Cathepsin S is implicated in: allergic disorders, including, but not limited to asthma; and allogeneic immune reponses, including, but not limited to, rejection of organ transplants or tissue grafts.

Another cysteine protease, Cathepsin F, has been found in macrophages and is involved in antigen processing. It is believed that Cathepsin F in stimulated lung macrophages and possibly other antigen presenting cells could play a role in airway inflammation (see G. P. Shi et al, J. Exp. Med. 191, 1177, 2000).

In view of the number of diseases wherein it is recognized that an increase in cysteine protease activity contributes to the pathology and/or symptomatology of the disease, molecules which inhibit the activity of this class of enzymes, in particular molecules which selectively inhibit Cathepsins B, H, K, L, O and S, are desirable as therapeutic agents. The present invention fulfills this and related needs.

### SUMMARY OF THE INVENTION

The present invention provides 3-(3,5-disubstituted-4-hydroxyphenyl)propionamide derivatives that selectively inhibit Cathepsin B. Pharmaceutical compositions comprising these compounds are useful in the treatment of diseases mediated by Cathepsin B.

Accordingly, in one aspect, the present invention is directed to a compound of Formula I: 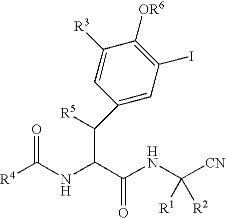
wherein:
R¹ and R² are independently hydrogen, alkyl, haloalkyl, hydroxyalkyl, aryl, or aralkyl; or
R¹ and R² together with the carbon atom to which they are attached form cycloalkyl or heterocycloalkyl;
R³ is alkyl or iodo; and
R⁴ is selected from the group consisting of aryl, heteroaryl, or heterocycloalkyl wherein R⁴ is optionally substituted with one, two or three R^{a} wherein:
   each R^{a} is independently selected from the group consisting of alkyl, alkoxy, hydroxy, alkylthio wherein the sulfur may be oxidized to sulfoxide or sulfone, halo, haloalkyl, haloalkoxy, nitro, amino, alkylamino, dialkylamino, aminocarbonyl, alkylaminocarbonyl, dialkylaminocarbonyl, aminosulfonyl, alkylaminosulfonyl, dialkylaminosulfonyl, aryl, heteroaryl, heterocycloalkyl, arylamino, heteroarylamino, heterocycloalkylamino, aryloxy, heteroaryloxy, heterocycloalkyloxy, arylthio wherein the sulfur may be oxidized to sulfoxide or sulfone, heteroarylthio wherein the sulfur may be oxidized to sulfoxide or sulfone, heterocycloalkylthio wherein the sulfur may be oxidized to sulfoxide or sulfone, cyano, acyl, carboxy, or alkoxycarbonyl wherein R^{a} is optionally substituted with one, two or three R^{b} wherein:
   each R^{b} is independently selected from the group consisting of alkyl, alkoxy, hydroxy, alkylthio wherein the sulfur may be oxidized to sulfoxide or sulfone, halo, haloalkyl, haloalkoxy, nitro, amino, alkylamino, dialkylamino, aminocarbonyl, alkylaminocarbonyl, dialkylaminocarbonyl, aminosulfonyl, alkylaminosulfonyl, dialkylaminosulfonyl, aryl, heteroaryl, heterocycloalkyl, arylamino, heteroarylamino, heterocycloalkylamino, aryloxy, heteroaryloxy, heterocycloalkyloxy, arylthio wherein the sulfur may be oxidized to sulfoxide or sulfone, heteroarylthio wherein the sulfur may be oxidized to sulfoxide or sulfone, heterocycloalkylthio wherein the sulfur may be oxidized to sulfoxide or sulfone, cyano, acyl, carboxy, or alkoxycarbonyl wherein each R^{b} is optionally substituted with one, two or three substituents independently selected from alkyl, alkoxy, hydroxy, alkylthio wherein the sulfur may be oxidized to sulfoxide or sulfone, halo, haloalkyl, haloalkoxy, carboxy, alkoxycarbonyl, amino, alkylamino, dialkylamino, aminocarbonyl, alkylaminocarbonyl, dialkylaminocarbonyl, aminosulfonyl, alkylaminosulfonyl, dialkylaminosulfonyl, cyano, or nitro;
R⁵ and R⁶ are independently hydrogen or alkyl;
   or a pharmaceutically acceptable salt thereof.

In a second aspect, this invention is directed to a pharmaceutical composition comprising a compound of Formula I, individual isomer, mixture of isomers or pharmaceutically acceptable salt thereof in admixture with one or more pharmaceutically suitable excipients.

In a third aspect, this invention is directed to a method of treating a disease in an animal in which inhibition of Cathepsin B, can prevent, inhibit or ameliorate the pathology and/or symptomatology of the disease, which method comprises administering to the animal a pharmaceutical composition comprising a therapeutically effective amount of compound of Formula I, an individual isomer, mixture of isomers or a pharmaceutically acceptable salt thereof. Preferably, the disease is cancer, rheumatoid arthritis, osteoarthritis, pneumocystis carinii, acute pancreatitis, inflammatory airway disease, bone and joint disorders, stroke, alcoholic hepatitis, cholestatic liver diseases, hepatitis C, and fatty liver diseases.

### DETAILED DESCRIPTION OF THE INVENTION

Definitions:

Unless otherwise stated, the following terms used in the specification and claims are defined for the purposes of this application and have the meanings given this section:

“Alkyl” means a straight or branched, saturated aliphatic radical having the number of carbon atoms indicated e.g., (C₁₋₆)alkyl includes methyl, ethyl, propyl, isopropyl, butyl, sec-butyl, isobutyl, tert-butyl, pentyl, hexyl, and the like. Preferably, methyl, ethyl, propyl, or isopropyl.

“Alkylene” means a straight or branched, saturated aliphatic divalent radical having one to six carbon atoms unless otherwise indicated e.g., methylene, ethylene, propylene, isopropylene, butylene, sec-butylene, isobutylene, tert-butylene, pentylene, hexylene, and the like. Preferably, methylene, ethylene, propylene, or isopropylene (including all its isomers).

“Aryl” means an aromatic monocyclic or bicyclic ring containing 6-12 carbon atoms unless otherwise indicated wherein each ring contained therein is comprised of 6 annular members e.g., (C₆₋₁₄)aryl includes phenyl, naphthalenyl, or anthracenyl, preferably phenyl.

“Aralkyl” means a radical -alkylene)-R where R is an aryl group as defined above, e.g., benzyl, phenylethyl, phenylpropyl, and the like.

“Animal” includes humans, non-human mammals (e.g., dogs, cats, rabbits, cattle, horses, sheep, goats, swine, deer, etc.) and non-mammals (e.g., birds, etc.).

“Alkylthio” means a radical —SR where R is alkyl as defined above, e.g., methylthio, ethylthio, propylthio (including all isomeric forms), butylthio (including all isomeric forms), and the like.

“Arylthio” means a radical —SR where R is aryl as defined above, e.g., phenylthio, napthylthio, and the like.

“Amino” means a radical —NH₂, or an N-oxide derivative, or a protected derivative thereof such as —NH→O, —NHBoc, —NHCBz, and the like.

“Arylamino” means a radical —NRR′ where R is hydrogen or alkyl and R′ is aryl as defined above, e.g., phenylamino, napthylamino, and the like.

“Acyl” means a radical —COR where R is alkyl, trifluoromethyl, aryl, heteroaryl, or heterocycloalkyl, e.g., methylcarbonyl, trifluoromethylcarbonyl, benzoyl, and the like.

“Alkylamino” means a radical —NHR where R is alkyl as defined above, e.g., methylamino, ethylamino, n-, iso-propylamino, n-, iso-, tert-butylamino, methylamino-N-oxide, and the like.

“Alkoxy” means a radical —OR where R is alkyl as defined above, e.g., methoxy, ethoxy, propoxy, or 2-propoxy, n-, iso-, or tert-butoxy, and the like.

“Aryloxy” means a radical —OR where R is aryl as defined above, e.g., phenoxy, napthyloxy, and the like.

“Alkoxycarbonyl” means a radical —COOR where R is alkyl as defined above, e.g., methoxycarbonyl, ethoxycarbonyl, n-propoxycarbonyl, or 2-propoxycarbonyl, n-, iso-, or tert-butoxycarbonyl, and the like.

“Aminocarbonyl” means a radical —CONH₂.

“Alkylaminocarbonyl” means a radical —CONHR where R is an alkyl group as defined above e.g, methylaminocarbonyl, ethylaminocarbonyl, and the like.

“Aminosulfonyl” means a radical —SO₂NH₂.

“Alkylaminosulfonyl” means a radical —SO₂NHR where R is an alkyl group as defined above e.g, methylaminosulfonyl, ethylaminosulfonyl, and the like.

“Cycloalkyl” means a cyclic saturated monovalent hydrocarbon radical of three to six carbon atoms, e.g., cyclopropyl, cyclobutyl, cyclopentyl, or cyclohexyl.

“Dialkylamino” means a radical —NRR′ where R and R′ are independently alkyl as defined above, or an N-oxide derivative, or a protected derivative thereof, e.g., dimethylamino, diethylamino, methylpropylamino, methylethylamino, n-, iso-, or tert-butylamino, and the like.

“Dialkylaminocarbonyl” means a radical —CONRR′ where R and R′ are independently an alkyl group as defined above e.g, dimethylaminocarbonyl, methylethylaminocarbonyl, and the like.

“Dialkylaminosulfonyl” means a radical —SO₂NRR′ where R and R′ are independently an alkyl group as defined above e.g, dimethylaminosulfonyl, methylethylaminosulfonyl, and the like.

“Disease” specifically includes any unhealthy condition of an animal or part thereof and includes an unhealthy condition, which may be caused by, or incident to, medical or veterinary therapy applied to that animal, i.e., the “side effects” of such therapy.

“Halo” means fluoro, chloro, bromo or iodo.

“Haloalkyl” means an alkyl group as defined herein wherein one, two, or three hydrogen atoms in the alkyl group has been replaced by a halo group as defined above, e.g., trifluoromethyl, difluorochloromethyl, tribromomethyl, chlorofluoroethyl, dichlorofluoroethyl, chlorodifluoromethyl including all the isomeric forms thereof, and the like.

“Haloalkoxy” means a radical —OR where R is haloalkyl as defined above, e.g., trifluoromethoxy, 2,2,2-trifluoroethoxy, and the like.

“Heteroaryl” means an aromatic monocyclic or bicyclic ring containing 5 to 9 ring atoms (unless otherwise indicated) wherein one, two, or three ring atoms are heteroatoms independently selected from N, O, or S(O)n (wherein n is 0, 1, or 2), the remaining ring atoms being carbon. Representative examples include, but are not limited to, thienyl, furanyl, pyrrolyl, imidazolyl, pyrimidinyl, pyradizinyl, pyrazinyl, isoxazolyl, oxazolyl, indolyl, benzo[b]thienyl, isobenzofuranyl, purinyl, quinolinyl, isoquinolyl, pterdinyl, perimidinyl, pyridyl, pyrazolyl, [2,4′]bipyridinylyl, 2-phenylpyridinyl, and the like, or tetrazolyl. The definition of heteroaryl also includes the N-oxide derivatives (≡N⁺→O⁻) i.e., where the nitrogen atom in the ring is oxidized.

“Heteroaralkyl” means a radical -(alkylene)-R where R is heteroaryl as defined above, e.g., pyridylmethyl, pyridylethyl, furanylmethyl, benzofuranylmethyl, and the like.

“Heteroarylamino” means a radical —NRR′ where R is hydrogen or alkyl and R′ is heteroaryl as defined above, e.g., pyridylamino, thienylamino, indolylamino, and the like.

“Heteroaryloxy” means a radical —OR where R is heteroaryl as defined above, e.g., pyridyloxy, thienyloxy, furanyloxy, and the like.

“Heteroarylthio” means a radical —SR where R is heteroaryl as defined above, e.g., pyridylthio, isoquinolinylthio, imidazolylthio and the like.

“Heterocycloalkyl” means a saturated or partially unsaturated mono or bicyclic ring containing three to ten ring atoms wherein one, two, or three of ring atoms are heteroatoms independently selected from N, O or S(O)ₙ (wherein n is 0, 1, or 2), the remaining ring atoms being carbon e.g., the term heterocycloalkyl includes tetrahydrofuranyl, piperidinyl, pyrrolidinyl, pyrrolinyl, imidazolidinyl, quinuclidinyl, morpholinyl, thiomorpholinyl, and the like. The definition of heterocycloalkyl also includes the N-oxide derivatives (≡N⁺→O⁻) i.e., where the nitrogen atom in the ring is oxidized.

“Heterocycloalkylalkyl” means a radical -(alkylene)-R where R is heterocycloalkyl as defined above, e.g., piperidinylmethyl, piperazinylethyl, pyrrolidinylmethyl, tetrahydrofuranylmethyl, and the like.

“Heterocycloalkylamino” means a radical —NRR′ where R is hydrogen or alkyl and R′ is heterocycloalkyl as defined above, e.g., tetrahydrofuranylamino, pyrrolidinylamino, and the like.

“Heterocycloalkyloxy” means a radical —OR where R is heterocycloalkyl as defined above, e.g., piperidinyloxy, piperazinyloxy, pyrrolidinyloxy, tetrahydrofuranyloxy, and the like.

“Heterocycloalkylthio” means a radical —SR where R is heterocycloalkyl as defined above, e.g., morpholinylthio, piperidinylthio, and the like.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Isomers” mean compounds of Formula I having identical molecular formulae but differ in the nature or sequence of bonding of their atoms or in the arrangement of their atoms in space. Isomers that differ in the arrangement of their atoms in space are termed “steroisomers”. Stereoisomers that are not mirror images of one another are termed “diastereomers” and stereoisomers that are nonsuperimposable mirror images are termed “enantiomers” or sometimes “optical isomers”. A carbon atom bonded to four nonidentical substituents is termed a “chiral center”. A compound with one chiral center has two enantiomeric forms of opposite chirality is termed a “racemic mixture”. A compound that has more than one chiral center has 2ⁿ⁻¹ enantiomeric pairs, where it is the number of chiral centers. Compounds with more than one chiral center may exist as ether an individual diasteromer or as a mixture of diastereomers, termed a “diastereomeric mixture”. When one chiral center is present a stereoisomer may be characterized by the absolute configuration of that chiral center. Absolute configuration refers to the arrangement in space of the substituents attached to the chiral center. Enantiomers are characterized by the absolute configuration of their chiral centers and described by the R- and S-sequencing rules of Cahn, Ingold and Prelog. Conventions for stereochemical nomenclature, methods for the determination of stereochemistry and the separation of stereoisomers are well known in the art (e.g., see “Advanced Organic Chemistry”, 3rd edition, March, Jerry, John Wiley & Sons, New York, 1985). It is understood that the names and illustration used in this Application to describe compounds of Formula I are meant to be encompassed all possible stereoisomers and any mixture, racemic or otherwise, thereof.

“Nitro” means the radical NO₂.

“Optional” or “optionally” means that the subsequently described event or circumstance may or may not occur, and that the description includes instances where the event or circumstance occurs and instances in which it does not. For example, the phrase “Ar is optionally substituted with —(C₁₋₆)alkyl” means that —(C₁₋₆)alkyl may but need not be present, and the description includes situations where the Ar group is substituted with —(C₁₋₆)alkyl and situations where the Ar group is not substituted with —(C₁₋₆)alkyl.

“Pathology” of a disease means the essential nature, causes and development of the disease as well as the structural and functional changes that result from the disease processes.

“Pharmaceutically acceptable” means that which is userul in preparing a pharmaceutical composition that is generall safe, non-toxic and neither biologically nor otherwise undesirabale and includes that which is acceptable for veterinary use as well as human pharmaceutical use.

“Pharmaceutically acceptable salts” means salts of compounds of Formula I which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartatic acid, citric acid, benzoic acid, o-(4-hydroxybenzoyl)benzoic acid, cinnamic acid, madelic acid, methanesulfonic acid, ethanesulfonic acid, 1,2-ethanedisulfonic acid, 2-hydroxyethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like.

Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

The present invention also includes the prodrugs of a compound of Formula I. Prodrugs means any compound which releases an active parent drug according to Formula I in vivo when such prodrug is administered to a mammalian subject. Prodrugs of a compound of Formula I are prepared by modifying functional groups present in the compound of Formula I in such a way that the modifications may be cleaved in vivo to release the parent compound. Prodrugs include compounds of Formula I wherein a hydroxy, amino, or sulfhydryl group in compound I is bonded to any group that may be cleaved in vivo to regenerate the free hydroxyl, amino, or sulfhydryl group, respectively. Examples of prodrugs include, but are not limited to esters (e.g., acetate, formate, and benzoate derivatives), carbamates (e.g., N,N-dimethylaminocarbonyl) of hydroxy functional groups in compounds of Formula I, and the like.

The present invention also includes the protected derivatives of a compound of Formula I. Protected derivatives means derivatives of compounds of Formula I in which a reactive site or sites are blocked with protective groups. Protected derivatives of compounds of Formula I are useful in the preparation of compounds of Formula I or in themselves may be active cysteine protease inhibitors. A comprehensive list of suitable protective groups can be found in T. W. Greene, Protective Groups in Organic Synthesis, John Wiley & Sons, Inc. 1981, the disclosure of which is incorporated herein by reference in its entirety.

The present invention also includes the N-oxide derivative of a compound of Formula I. N-oxide derivative of a compound of Formula I can form when a compound of Formula I carries a nitrogen atom at a position that is susceptible to oxidation.

“Therapeutically effective amount” means that amount which, when administered to an animal for treating a disease, is sufficient to effect such treatment for the disease.

“Treatment” or “teating” means any administration of a compound of the present invention and includes:
(1) preventing the disease from occurring in an animal, which may be predisposed to the disease but does not yet experience or display the pathology or symptomatology of the disease,
(2) inhibiting the disease in an animal that is experiencing or displaying the pathology or symptomatology of the diseased (i.e., arresting further development of the pathology and/or symptomatology), or
(3) ameliorating the disease in an animal that is experiencing or displaying the pathology or symptomatology of the diseased (i.e., reversing the pathology and/or symptomatology).

Representative compounds of Formula I where R⁵ is hydrogen and other groups are as defined below are:

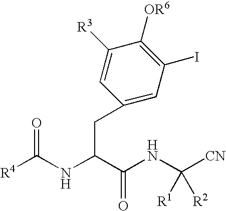

| Cpd # | R¹ | R² | R¹ + R² | R³ | R⁴ | R⁶ |
|---|---|---|---|---|---|---|
| 1 | H | H | | I | 4-(2-pyridin-4-ylami- | H |
| | | | | | no-thiazol-4-yl)phenyl | |
| 2 | H | H | | I | 4-morpholin-4-ylphenyl | H |
| 3 | H | H | | I | morpholin-4-yl | H |
| 4 | | | cyclo- | I | 4-morpholin-4-ylphenyl | H |
| | | | propyl | | | |
| 5 | H | H | | I | 4-morpholin-4-ylphenyl | CH₃ |
| 6 | H | H | | I | 4-[2-(4-methylpipera- | H |
| | | | | | zin-1-yl)-thia- | |
| | | | | | zol-4-yl]phenyl | |
| 7 | H | H | | CH₃ | 4-morpholin-4-ylphenyl | H |
| 8 | H | H | | CH₂CH₃ | 4-morpholin-4-ylphenyl | H |

### Presently Preferred Embodiments

While the broadest definition of this Invention is set forth in the Summary of the Invention, certain aspects of the Invention are preferred.
(A) One preferred group of compounds is that wherein R¹ and R² are hydrogen.
(B) Another preferred group of compounds is that wherein R¹ and R² form cycloalkyl, preferably cyclopropyl.
(C) Another preferred group of compounds is that wherein R¹ and R² form heterocycloalkyl, preferably piperidin-4-yl, 1-alkylpiperidin-4-yl (preferably, 1-methylpiperidin-4-yl), morpholin-4-yl, pyrrolidinyl, azetidinyl, tetrahydrofuranyl, oxetanyl, azocanyl, oxocanyl, 1,3-, 1,4-, or 1,5-diazocanyl, 1,3-, 1,4-, or 1,5-dioxocanyl, 1,3-, 1,4-, or 1,5-oxazocanyl, 1,3-, 1,4-, or 1,5-diazepanyl, 1,3-, 1,4-, or 1,5-dioxepanyl, 1,3-, 1,4-, or 1,5-oxazepanyl, tetrahydrothiophenyl, hexahydropyrimidinyl, hexahydropyridazinyl, 1,4,5,6-tetrahydropyrimidinyl, pyrazolidinyl, dihydrooxazolyl, dihydrothiazolyl, dihydroimidazolyl, isoxazolinyl, oxazolidinyl, thiomorpholinyl, thiothiomorphlinyl 1,1-dioxide, imidazolidinyl, dioxanyl, or tetrahydropyridinyl.
(D) Another preferred group of compounds is that wherein R¹ is hydrogen and R² is haloalkyl.
(E) Another preferred group of compounds is that wherein R¹ is hydrogen and R² is hydroxyalkyl.

Within the above groups (A)-(E), a more preferred group of compounds is that wherein:
R⁵ is hydrogen or methyl, preferably hydrogen; and
R⁶ is hydrogen or methyl, preferably hydrogen.

Within the above more preferred group, an even more preferred group of compounds is that wherein:
R³ is alkyl, preferably methyl, ethyl, or propyl.

Within the above more preferred group, another even more preferred group of compounds is that wherein:
R³ is iodo.

Within the above preferred, more preferred, and even more preferred groups, particularly preferred group of compounds are those wherein:
R⁴ is aryl, heteroaryl, or heterocyloalkyl optionally substituted with one, two or three R^{a} wherein:
each R^{a} is independently selected from the group consisting of alkyl, alkoxy, hydroxy, alkylthio wherein the sulfur may be oxidized to sulfoxide or sulfone, halo, haloalkyl, haloalkoxy, nitro, amino, alkylamino, dialkylamino, aminocarbonyl, alkylaminocarbonyl, dialkylaminocarbonyl, aminosulfonyl, alkylaminosulfonyl, dialkylaminosulfonyl, aryl, heteroaryl, heterocycloalkyl, arylamino, heteroarylamino, heterocycloalkylamino, aryloxy, heteroaryloxy, heterocycloalkyloxy, arylthio wherein the sulfur may be oxidized to sulfoxide or sulfone, heteroarylthio wherein the sulfur may be oxidized to sulfoxide or sulfone, heterocycloalkylthio wherein the sulfur may be oxidized to sulfoxide or sulfone, cyano, acyl, carboxy, or alkoxycarbonyl wherein R^{a} is optionally substituted with one, two or three R^{b} wherein:
each R^{b} is independently selected from the group consisting of alkyl, alkoxy, hydroxy, alkylthio wherein the sulfur may be oxidized to sulfoxide or sulfone, halo, haloalkyl, haloalkoxy, nitro, amino, alkylamino, dialkylamino, aminocarbonyl, alkylaminocarbonyl, dialkylaminocarbonyl, aminosulfonyl, alkylaminosulfonyl, dialkylaminosulfonyl, cyano, acyl, carboxy, or alkoxycarbonyl.

Another particularly preferred group of compounds are those wherein:
R⁴ is selected from the group consisting of aryl, heteroaryl, or heterocycloalkyl wherein R⁴ is optionally substituted with one, two or three R^{a} wherein:
   each R^{a} is independently selected from the group consisting of alkyl, alkoxy, hydroxy, alkylthio wherein the sulfur may be oxidized to sulfoxide or sulfone, halo, haloalkyl, haloalkoxy, nitro, amino, alkylamino, dialkylamino, aminocarbonyl, alkylaminocarbonyl, dialkylaminocarbonyl, aminosulfonyl, alkylaminosulfonyl, dialkylaminosulfonyl, aryl, heteroaryl, heterocycloalkyl, arylamino, heteroarylamino, heterocycloalkylamino, aryloxy, heteroaryloxy, heterocycloalkyloxy, arylthio wherein the sulfur may be oxidized to sulfoxide or sulfone, heteroarylthio wherein the sulfur may be oxidized to sulfoxide or sulfone, heterocycloalkylthio wherein the sulfur may be oxidized to sulfoxide or sulfone, cyano, acyl, carboxy, or alkoxycarbonyl provided that R⁴ is substituted with at least one R^{a} that is an aryl, heteroaryl or heterocycloalkyl ring or a group that has an aryl, heteroaryl or heterocyclic ring and further wherein R^{a} is optionally substituted with one, two or three R^{b} wherein:
   each R^{b} is independently selected from the group consisting of alkyl, alkoxy, hydroxy, alkylthio wherein the sulfur may be oxidized to sulfoxide or sulfone, halo, haloalkyl, haloalkoxy, nitro, amino, alkylamino, dialkylamino, aminocarbonyl, alkylaminocarbonyl, dialkylaminocarbonyl, aminosulfonyl, alkylaminosulfonyl, dialkylaminosulfonyl, aryl, heteroaryl, heterocycloalkyl, arylamino, heteroarylamino, heterocycloalkylamino, aryloxy, heteroaryloxy, heterocycloalkyloxy, arylthio wherein the sulfur may be oxidized to sulfoxide or sulfone, heteroarylthio wherein the sulfur may be oxidized to sulfoxide or sulfone, heterocycloalkylthio wherein the sulfur may be oxidized to sulfoxide or sulfone, cyano, acyl, carboxy, or alkoxycarbonyl provided that R^{a} is substituted with at least one R^{b} that is an aryl, heteroaryl or heterocycloalkyl ring or a group that has an aryl, heteroaryl or heterocyclic ring wherein each R^{b} is optionally substituted with one, two or three substituents independently selected from alkyl, alkoxy, hydroxy, alkylthio wherein the sulfur may be oxidized to sulfoxide or sulfone, halo, haloalkyl, haloalkoxy, carboxy, alkoxycarbonyl, amino, alkylamino, dialkylamino, aminocarbonyl, alkylaminocarbonyl, dialkylaminocarbonyl, aminosulfonyl, alkylaminosulfonyl, dialkylaminosulfonyl, cyano, or nitro.
      (F) Another preferred group of compounds is that wherein R¹ and R² are hydrogen; and
R⁴ is selected from the group consisting of aryl, heteroaryl, or heterocycloalkyl wherein R⁴ is optionally substituted with one, two or three R^{a} wherein:
   each R^{a} is independently selected from the group consisting of alkyl, alkoxy, hydroxy, alkylthio wherein the sulfur may be oxidized to sulfoxide or sulfone, halo, haloalkyl, haloalkoxy, nitro, amino, alkylamino, dialkylamino, aminocarbonyl, alkylaminocarbonyl, dialkylaminocarbonyl, aminosulfonyl, alkylaminosulfonyl, dialkylaminosulfonyl, aryl, heteroaryl, heterocycloalkyl, arylamino, heteroarylamino, heterocycloalkylamino, aryloxy, heteroaryloxy, heterocycloalkyloxy, arylthio wherein the sulfur may be oxidized to sulfoxide or sulfone, heteroarylthio wherein the sulfur may be oxidized to sulfoxide or sulfone, heterocycloalkylthio wherein the sulfur may be oxidized to sulfoxide or sulfone, cyano, acyl, carboxy, or alkoxycarbonyl provided that R⁴ is substituted with at least one R^{a} that is an aryl, heteroaryl or heterocycloalkyl ring or a group that has an aryl, heteroaryl or heterocyclic ring and further wherein R^{a} is optionally substituted with one, two or three R^{b} wherein:
each R^{b} is independently selected from the group consisting of alkyl, alkoxy, hydroxy, alkylthio wherein the sulfur may be oxidized to sulfoxide or sulfone, halo, haloalkyl, haloalkoxy, nitro, amino, alkylamino, dialkylamino, aminocarbonyl, alkylaminocarbonyl, dialkylaminocarbonyl, aminosulfonyl, alkylaminosulfonyl, dialkylaminosulfonyl, aryl, heteroaryl, heterocycloalkyl, arylamino, heteroarylamino, heterocycloalkylamino, aryloxy, heteroaryloxy, heterocycloalkyloxy, arylthio wherein the sulfur may be oxidized to sulfoxide or sulfone, heteroarylthio wherein the sulfur may be oxidized to sulfoxide or sulfone, heterocycloalkylthio wherein the sulfur may be oxidized to sulfoxide or sulfone, cyano, acyl, carboxy, or alkoxycarbonyl provided that R^{a} is substituted with at least one R^{b} that is an aryl, heteroaryl or heterocycloalkyl ring or a group that has an aryl, heteroaryl or heterocyclic ring wherein each R^{b} is optionally substituted with one, two or three substituents independently selected from alkyl, alkoxy, hydroxy, alkylthio wherein the sulfur may be oxidized to sulfoxide or sulfone, halo, haloalkyl, haloalkoxy, carboxy, alkoxycarbonyl, amino, alkylamino, dialkylamino, aminocarbonyl, alkylaminocarbonyl, dialkylaminocarbonyl, aminosulfonyl, alkylaminosulfonyl, dialkylaminosulfonyl, cyano, or nitro.

Within this group, a more preferred group of compounds is that wherein R⁵ and R⁶ are hydrogen and R³ is iodo.

### General Synthetic Scheme

Compounds of this invention can be made by the methods discussed below.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure.

The starting materials and the intermediates of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., more preferably from about 0° C. to about 125° C. and most preferably at about room (or ambient) temperature, e.g., about 20° C.

Compounds of Formula I can be prepared by methods described and illustrated in Schemes 1 and 2 below.

A compound of Formula I where R¹, R², R³, R⁴, R⁵ and R⁶ are as described in the Summary of the Invention can be prepared as shown in Scheme 1 below.

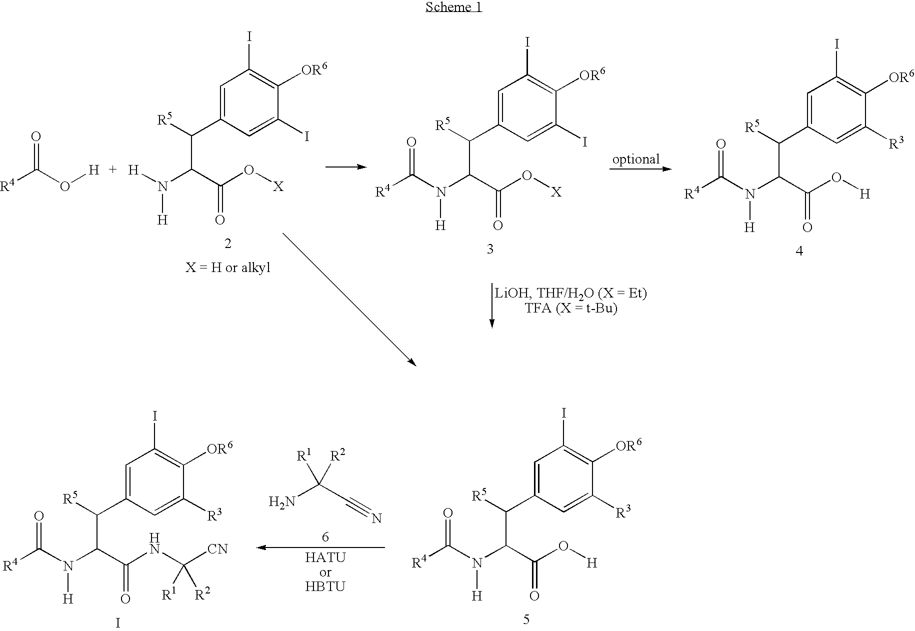

Reaction of an acid of formula 1 with a 3,5-diiodotyrosine derivative of formula 2 where X is hydrogen or alkyl (preferably methyl, ethyl, or tert-butyl) provides a compound of formula 3 or 5. The reaction is carried out in the presence of a coupling agent such as HATU, EDC/HOBt, and the like to provide a compound of formula 3. Suitable organic solvents for the above reactions are polar organic solvents such as tetrahydrofuran, dioxane, dimethylformamide, and the like.

Alternatively, a compound of formula 2 where X is alkyl can be reacted with an acid derivative e.g., an acid halide, of a compound of formula 1 in the presence of a base such as triethylamine, pyridine, and the like to provide a compound of formula 3.

Compounds of formula 1 such as benzoic acid, napthoic acid, nicotinic acid, 4-morpholin-4-ylbenzoic acid and isoincotinic acid are commercially available. Other compounds of formula 1 can be prepared by methods disclosed in PCT patent applications publication No. WO 00/55126, U.S. Pat. No. 6,353,601, and Applicants PCT patent application No. US 02/06533, the disclosures of which are incorporated herein by reference in their entirety. Compounds of formula 2 such as 3,5-diiodotyrosine are commercially available. Others can be prepared by methods well known in the art. For example, 3,5-diiodotyrosine can be converted to its corresponding alkyl ester (X=alkyl) by it in the desired alcohol such as methanol, ethanol, and the like in the presence of an acid such as hydrochloric acid.

A compound of formula 3 can optionally be converted to a compound of formula 4 where R³ is alkyl by reacting 3 with alkyltin chloride. The reaction is carried out in aqueous base such as aqueous potassium hydroxide, and the like and in the presence of a palladium catalyst such as palladium II chloride to give a mixture of dialkylated and the monoalkylated products. The desired monoalkylated product is isolated by column chromatography. Alternatively, a compound of formula 4 where R³ is ethyl can be prepared by the procedure described in working example below.

Hydrolysis of the ester group in 3 under acidic (X=tert-butyl) or basic (X=methyl or ethyl) hydrolysis reaction conditions provides a compound of formula 5. Reaction of 4 or 5 with an aminoacetonitrile compound of formula 6 where R¹ and R² are as defined in the Summary of the Invention then provides a compound of Formula I. The reaction is typically carried out in the presence of a coupling agent such as HATU or HBTU. Compounds of formula 6 such as 2-aminoacetonitrile are commercially available or they can be prepared by methods well known in the art. Some such methods are described in WO 00/55126 and Applicants PCT patent application No. US 02/06533 the disclosures of which are incorporated herein by reference in its entirety.

Alternatively, a compound of Formula I where R¹, R², R³, R⁴, R⁵ and R⁶ are as described in the Summary of the Invention can be prepared as shown in Scheme 2 below.

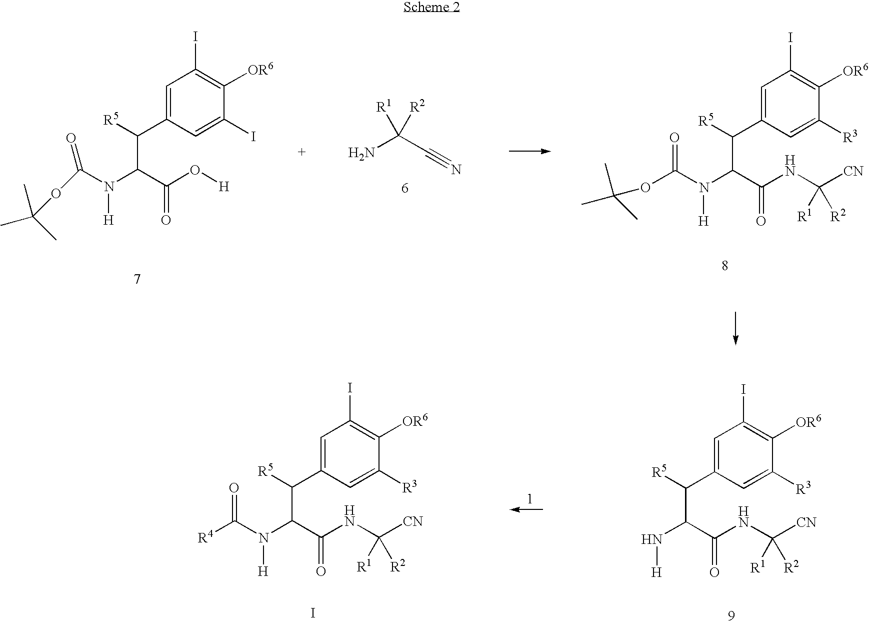

Reaction of a compound of formula 7 with an aminoacetonitrile of formula 6 under the reaction conditions described in Scheme 1 above, provides a compound of formula 8. Compound 7 can be readily prepared by reacting the corresponding amino acid with BOC anhydride in the presence of a base such as sodium hydroxide, and the like.

Removal of the BOC group is carried out under acidic hydrolysis reaction conditions utilizing acids such as methanesulfonic acid, and the like and in a suitable organic solvent such as tetrahydrofuran, and the like.

Compound 9 is then coupled with a compound of formula 1 under the reaction conditions described above to provide a compound of Formula I.

Detailed descriptions of synthesis of a compound of Formula I by the above procedures are provided in working examples below.

Additional Processes for Preparing Compounds of Formula I:

Compounds of Formula I can also be prepared by modification of a group present on a corresponding compound of Formula I. For example, a compound of Formula I where R⁶ is substituted with hydrogen can be alkylated with a suitable alkylating agent such as trialkyllsilyldiazomethane to provide a compound of Formula I where R⁶ is alkyl.

A compound of Formula I can be prepared as a pharmaceutically acceptable acid addition salt by reacting the free base form of the compound with a pharmaceutically acceptable inorganic or organic acid. Alternatively, a pharmaceutically acceptable base addition salt of a compound of Formula I can be prepared by reacting the free acid form of the compound with a pharmaceutically acceptable inorganic or organic base. Inorganic and organic acids and bases suitable for the preparation of the pharmaceutically acceptable salts of compounds of Formula I are set forth in the definitions section of this application. Alternatively, the salt forms of the compounds of Formula I can be prepared using salts of the starting materials or intermediates.

The free acid or free base forms of the compounds of Formula I can be prepared from the corresponding base addition salt or acid addition salt form. For example, a compound of Formula I in an acid addition salt form can be converted to the corresponding free base by treating with a suitable base (e.g., ammonium hydroxide solution, sodium hydroxide, etc.). A compound of Formula I in a base addition salt form can be converted to the corresponding free acid by treating with a suitable acid (e.g., hydrochloric acid, etc).

The N-oxides of compounds of Formula I can be prepared by methods known to those of ordinary skill in the art. For example, N-oxides can be prepared by treating an unoxidized form of the compound of Formula I with an oxidizing agent (e.g., trifluoroperacetic acid, permaleic acid, perbenzoic acid, peracetic acid, meta-chloroperoxybenzoic acid, etc.) in a suitable inert organic solvent (e.g., a halogenated hydrocarbon such as methylene chloride) at approximately 0 C. Alternatively, the N-oxides of the compounds of Formula I can be prepared from the N-oxide of an appropriate starting material.

Compounds of Formula I in unoxidized form can be prepared from N-oxides of compounds of Formula I by treating with a reducing agent (e.g., sulfur, sulfur dioxide, triphenyl phosphine, lithium borohydride, sodium borohydride, phosphorus trichloride, tribromide, etc.) in a suitable inert organic solvent (e.g., acetonitrile, ethanol, aqueous dioxane, etc.) at 0 to 80 C.

Prodrugs of the compounds of Formula I can be prepared by methods known to those of ordinary skill in the art (e.g., for further details see Saulnier et al. (1994), Bioorganic and Medicinal Chemistry Letters. 4:1985). For example, appropriate prodrugs can be prepared by reacting a non-derivatized compound of Formula I with a suitable carbamylating agent (e.g., 1,1-acyloxyalkylcarbonochloridate, para-nitrophenyl carbonate, etc.).

Protected derivatives of the compounds of Formula I can be made by means known to those of ordinary skill in the art. A detailed description of the techniques applicable to the creation of protective groups and their removal can be found in T. W. Greene, Protective Groups in Organic Synthesis, John Wiley & Sons, Inc. 1981.

Compounds of Formula I can be prepared as their individual stereoisomers by reacting a racemic mixture of the compound with an optically active resolving agent to form a pair of diastereoisomeric compounds, separating the diastereomers and recovering the optically pure enantiomer. While resolution of enantiomers can be carried out using covalent diasteromeric derivatives of compounds of Formula I, dissociable complexes are preferred (e.g., crystalline diastereoisomeric salts). Diastereomers have distinct physical properties (e.g., melting points, boiling points, solubilities, reactivity, etc.) and can be readily separated by taking advantage of these dissimilarities. The diastereomers can be separated by chromatography or, preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques Andre Collet, Samuel H. Wilen, Enantiomers, Racemates and Resolutions, Honh Wiley & Sons, Inc. (1981).

### Pharmacology and Utility

The compounds of this invention are Cathepsin B inhibitors, and are useful for treating diseases in which Cathepsin B activity contributes to the pathology and/or symptomatology e.g., cancer (see Michaud, S.; Gour, B. Exp. Opin. Ther. Pat. 1998, 8, 645, Koblinski, J. E. et al. Clinica Chim. Acta 2000, 291, 113, Berquin, I. M. and Sloane, BF Adv. Exp. Med. Biol. 1996, 389, 281, and Szpaderska, A. M. and Frankfater, A. Cancer Res. 2001, 61, 3493); neurodegenerative disorders (see Petanceska, S. et al. Neuroscience 1994, 59, 729); stroke (see Seyfried, D. M. et al. Brain Res. 2001, 901, 94); ischemia, rheumatoid arthritis (see Keyszer, G. et al. Arthritis Rheum. 1993, 41, 1378, Esser, R. E. et al. Arthritis Rheum. 1994, 37, 236, and Hashimoto, Y. et al. Biochem Biophys. Res. Commun. 2001, 283, 334); osteoarthritis (see Lang, A. et al. J. Rheumatol. 2000, 27, 1971); acute pancreatitis (see Halangk, W et al. J. Clin. Invest. 2000, 106, 773); liver disease (see Roberts, L. R. et al. Gastroenterology 1997, 113, 1714, Jones, B. A. et al. Am. J. Physiol. 1997, 272, G1109, Faubion, W. A. et al. J. Clin. Invest. 1999, 103, 137, Roberts, L. R. et al. Cell Biochem. Biophys. 1999, 30, 71, Guicciardi, M. E. et al. J. Clin. Invest. 2000, 106, 1127, Guicciardi, M. E. et al. Hepatology 2001, 34, 844, and Guicciardi, M. E. et al. Am. J. Physiol 2001, 159, 2045); atherosclerosis (see Chen, J et al Circulation 2002, 105, 2766 and Li, W. et al Arterioscler. Thromb. Vasc. Biol. 2001, 21, 1124); Alzheimer's disease (see Tagawa, K. T. et al Biochem. Biophys. Res. Commun. 1991, 177, 377 and Cataldo, A. M. et al. Brain Res. 1990, 513, 181); and periodontal disease (see Eley, B. M. and Cox, S. W. J. Periodontal Res. 1996, 31, 381).

### Testing

The Cathepsin B inhibitory activities of the compounds of the invention can be determined by methods known to those of ordinary skill in the art. Suitable in vitro assays for measuring protease activity and the inhibition thereof by test compounds are known. Typically, the assay measures protease induced hydrolysis of a peptide based substrate. Details of assays for measuring protease inhibitory activity are set forth in Biological Examples 1 below.

### Administration and Pharmaceutical Compositions

In general, compounds of Formula I will be administered in therapeutically effective amounts via any of the usual and acceptable modes known in the art, either singly or in combination with another therapeutic agent. A therapeutically effective amount may vary widely depending on the severity of the disease, the age and relative health of the subject, the potency of the compound used and other factors. For example, therapeutically effective amounts of a compound of Formula I for anticoagulant therapy may range from 0.1 micrograms per kilogram body weight (μg/kg) per day to 10 milligram per kilogram body weight (mg/kg) per day, typically 1 μg/kg/day to 0.1 mg/kg/day. Therefore, a therapeutically effective amount for a 80 kg human patient may range from 10 μg/day to 10 mg/day, typically 0.1 mg/day to 10 mg/day. In general, one of ordinary skill in the art, acting in reliance upon personal knowledge and the disclosure of this Application, will be able to ascertain a therapeutically effective amount of a compound of Formula I for treating a given disease.

The compounds of Formula I can be administered as pharmaceutical compositions by one of the following routes: oral, systemic (e.g., transdermal, intranasal or by suppository) or parenteral (e.g., intramuscular, intravenous or subcutaneous). Compositions can take the form of tablets, pills, capsules, semisolids, powders, sustained release formulations, solutions, suspensions, elixirs, aerosols, or any other appropriate composition and are comprised of, in general, a compound of Formula I in combination with at least one pharmaceutically acceptable excipient. Acceptable excipients are non-toxic, aid administration, and do not adversely affect the therapeutic benefit of the active ingredient. Such excipient may be any solid, liquid, semisolid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk, and the like. Liquid and semisolid excipients may be selected from water, ethanol, glycerol, propylene glycol and various oils, including those of petroleum, animal, vegetable or synthetic origin (e.g., peanut oil, soybean oil, mineral oil, sesame oil, etc.). Preferred liquid carriers, particularly for injectable solutions, include water, saline, aqueous dextrose and glycols.

The amount of a compound of Formula I in the composition may vary widely depending upon the type of formulation, size of a unit dosage, kind of excipients and other factors known to those of skill in the art of pharmaceutical sciences. In general, a composition of a compound of Formula I for treating a given disease will comprise from 0.01% w to 10% w, preferably 0.3% w to 1% w, of active ingredient with the remainder being the excipient or excipients. Preferably the pharmaceutical composition is administered in a single unit dosage form for continuous treatment or in a single unit dosage form ad libitum when relief of symptoms is specifically required. Representative pharmaceutical formulations containing a compound of Formula I are described in Formulation Examples 1-3 below.

### EXAMPLES

The following preparations and examples are given to enable those skilled in the art to more clearly understand and to practice the present invention. They should not be considered as limiting the scope of the invention, but merely as being illustrative and representative thereof.

### Example 1

### Synthesis of (S)-N-[1-(cyanomethylcarbamoyl)-2-(4-hydroxy-3,5-diiodophenyl)ethyl]-4-morpholin-4-yl-benzamide (compound 2)

Step 1

4-Morpholinobenzoic acid hydrochloride (0.488 g, 2.0 mmol), HOBT (0.297 g, 2.2 mmol), and triethylamine (0.84 mL, 6.0 mmol) were stirred at room temperature with dry N,N-dimethylformamide (DMF) (20 mL). EDC (0.460 g, 2.4 mmol) was added. After 30 min., a solution of L-3,5-diiodotyrosine (0.902 g, 2.0 mmol) in DMF (10.0 mL), triethylamine (0.84 mL, 6.0 mmol), and water (1.5 mL) was added. Stirring was continued for 16 hours. The solvent was then evaporated off and the residue was partitioned between dichloromethane and 1N HCl. The organic phase was separated and dried over anhydrous magnesium sulfate. Filtration and solvent evaporation gave 816 mg of the crude (S)-3-(4-hydroxy-3,5-diiodo-phenyl)-2-(4-morpholin-4-yl-benzoylamino)-propionic acid that was used without purification for the following reaction.

Step 2

(S)-3-(4-Hydroxy-3,5-diiodo-phenyl)-2-(4-morpholin-4-yl-benzoylamino)-propionic acid (0.79 g), aminoacetonitrile hydrochloride (0.129 g, 1.27 mmol), HBTU (0.482 g, 1.27 mmol) and N-methylmorpholine (0.70 mL, 6.35 mmol) were dissolved in DMF (20 mL) and stirred overnight. The solvent was evaporated and the residue was partitioned between 0.5 N HCl and dichloromethane in a separatory funnel. The organic phase was separated, washed with water, saturated sodium bicarbonate, and brine, and then dried over magnesium sulfate. Filtration and solvent evaporation gave 480 mg of residue that was flash chromatographed on silica gel, eluting with Mar. 7, 1990 (v/v/v) methanol/acetone/dichloromethane gave the title compound (115 mg) as a pink powder. Proton NMR (270 MHz, CDCl₃): δ 9.33 (bs, 1H), δ 8.77 (t, J=6 Hz, 1H), δ 8.40 (d, J=8 Hz, 1H), δ 7.74 (m, 4H), δ 6.95 (d, J=9 Hz, 2H), δ 4.55 (m, 1H), δ 4.18 (d, J=6 Hz, 2H), δ 3.73 (t, J=4 Hz, 4H), δ 3.21 (t, J=4 Hz, 4H), δ 2.68-2.97 (m, 2H). LCMS (electrospray) mH⁺ 661 (100%).

Proceeding as described in Example 1 above, but substituting appropriate starting materials provided the following compounds of Formula I.

(S)-N-[1-(Cyanomethylcarbamoyl)-2-(4-hydroxy-3,5-diiodophenyl)-ethyl]4-[2-(pyridin-4-ylamino)-thiazol-4-yl]-benzamide trifluoroacetate (compound 1). Proton NMR (270 MHz, DMSO-d6): δ 12.05 (s, 1H), δ 9.36 (bs, 1H), δ 8.88 (t, J=5 Hz, 1H), δ 8.63 (d, J=6 Hz, 2H), δ 8.09 (d, J=7 Hz, 4H), δ 7.94 (m, 3H), δ 7.77 (s, 2H), δ 4.62 (m, 1H), δ 4.21 (d, J=5 Hz, 2H), δ 2.8-3.05 (m, 2H). LCMS (electrospray): mH⁺ 751 (100%).

(S)-Morpholine-4-carboxylic acid [1-(cyanomethyl-carbamoyl)-2-(4-hydroxy-3,5-diiodo-phenyl)-ethyl]-amide (compound 3). Proton NMR (270 MHz, DMSO-d6): δ 9.39 (bs, 1H), δ 8.66 (t, J=5 Hz, 1H), δ 7.61 (s, 2H), δ 6.72 (d, J=9 Hz, 1H), δ 4.2 (m, 3H), δ 3.51 (t, J=5 Hz, 4H), δ 3.26 (m, 4H), δ 2.6-2.9 (m, 2H). LCMS (electrospray): mH⁺ 585 (100%).

(S)-N-[1-(1-Cyano-cyclopropylcarbamoyl)-2-(4-hydroxy-3,5-diiodo-phenyl)-ethyl]4-morpholin-4-yl-benzamide (compound 4). Proton NMR (270 MHz, DMSO-d6): δ 9.35 (bs, 1H), δ 9.00 (s, 1H), δ 8.34 (d, J=8 Hz, 1H), δ 7.78 (d, J=9 Hz, 2H), δ 7.68 (s, 2H), δ 6.96 (d, J=9 Hz, 2H), δ 4.46 (m, 1H), δ 3.73 (bt, 4H), δ 3.22 (bt, 4H), δ 2.85 (m, 2H), δ 1.48 (bs, 2H), δ 1.06 (bs, 2H). LCMS (electrospray): mH⁺ 687 (100%).

### Example 2

### Synthesis of (S)-N-[1-(cyanomethylcarbamoyl)-2-(3,5-diiodo-4-methoxyphenyl)ethyl]4-morpholin-4-yl-benzamide (compound 5)

(S)-N-[1-(Cyanomethyl-carbamoyl)-2-(4-hydroxy-3,5-diiodophenyl)-ethyl]4-morpholin-4-yl-benzamide (0.050 g, 0.076 mmol) was stirred with methanol (10 mL) and acetonitrile (10 mL). Trimethylsilyldiazomethane in hexanes (0.76 mL, 2.0 M, 0.15 mmol) was added with stirring. After 2 hours the solvent was evaporated and the residue was chromatographed on silica gel. Elution with 3/7/90 methanol/acetone/dichloromethane provided the title compound (31.2 mg) as an off-white solid (a 61% yield).

Proton NMR (270 MHz, DMSO-d6): δ 8.77 (t, J=5 Hz, 1H), δ 8.44 (d, J=8 Hz, 1H), δ 7.83 (s, 2H), δ 7.72 (d, J=9 Hz, 2H), δ 6.95 (d, J=9 Hz, 2H), δ 4.58 (m, 1H), δ 4.17 (m, 2H), δ 3.73 (br, 4H), δ 3.68 (s, 3H), δ 3.20 (bt, 4H), δ 2.8-3.0 (m, 2H). LCMS (electrospray): mH⁺ 675 (100%).

### Example 3

### Synthesis of (S)-N-[1-(cyanomethyl-carbamoyl)-2-(4-hydroxy-3,5-diiodo-phenyl)-ethyl]-4-[2-(4-methyl-piperazin-1-yl)-thiazol-4-yl]-benzamide (compound 6)

Step 1

2-tert-Butoxycarbonylamino-3-(4-hydroxy-3,5-diiodophenyl)-propionic acid 2,5-dioxo-pyrrolidin-1-yl ester (1.00 g, 1.59 mmol), available from Bachem, aminoacetonitrile (176 mg, 1.90 mmol), and N-methylmorpholine (0.42 mL, 3.8 mmol) were stirred at room temperature in 25 mL dry acetonitrile. After 16 h, the solvent was rotary evaporated and the residue was partitioned between ethyl acetate and 0.5 N aqueous HCl in a separatory funnel. The organic phase was washed with water, and brine, and dried over anhydrous magnesium sulfate. Filtration and solvent evaporation afforded 450 mg of [1-(cyanomethylcarbamoyl)-2-(4-hydroxy-3,5-diiodophenyl)ethyl]-carbamic acid tert-butyl ester as a yellow solid (50%). This was used without purification for the subsequent step.

Step 2

To a solution of (S)-[1-(cyanomethylcarbamoyl)-2-(4-hydroxy-3,5-diiodophenyl)ethyl]-carbamic acid tert-butyl ester (1.88 g, 3.30 mmol) in 100 mL anhydrous THF was added anhydrous methanesulfonic acid (1.20 mL, 16.5 mmol). After 17 hours the volume of the reaction mixture was reduced by half. The remaining solution was vigorously stirred while diethyl ether was added. The white precipitate was collected, rinsed with anhydrous ether, dried briefly to give (S)-2-amino-N-cyanomethyl-3-(4-hydroxy-3,5-diiodo-phenyl)-propionamide methanesulfonate (1.14 g), which was used immediately for the subsequent coupling without further purification.

Step 3

To a mixture of 4-[2-(4-methyl-piperazin-1-yl)-thiazol-4-yl]-benzoic acid hydrobromide (prepared as described in PCT patent applications publication No. WO 00/55126) (0.680 g, 1.76 mmol), (S)-2-amino-N-cyanomethyl-3-(4-hydroxy-3,5-diiodo-phenyl)-propionamide methanesulfonate (1.00 g, 1.76 mmol), and HATU (0.671 g, 1.76 mmol) was added triethylamine (1.47 mL, 10.6 mmol). The reaction mixture was stirred for 2 hours and then the solvent was removed by evaporation. The residue was taken up in a minimum amount of 15% (v/v) methanol in dichloromethane and stirred while diethyl ether was added. The precipitate was collected by filtration, washed with more ether, and dried. Flash chromatography of this material was carried out on silica gel, applying the sample with 50% methanol in dichloromethane and eluting the column with 10% methanol in dichloromethane to give the title compound (426 mg) as a pink solid (a 32% yield). Proton NMR (270 MHz, CDCl₃): δ 8.83 (t, J=6 Hz, 1H), δ 8.69 (d, J=8 Hz, 1H), δ 7.94 (d, J=8 Hz, 2H), δ 7.82 (d, J=8 Hz, 2H), δ 7.75 (s, 2H), δ 7.45 (s, 1H), δ 4.61 (m, 1H), δ 4.18 (d, J=6 Hz, 2H), δ 3.47 (bt, 4H), δ 2.75-3.05 (m, 2H), δ 2.45 (bt, 4H), δ 2.24 (s, 3H). LCMS (electrospray) mH⁺ 757 (100%).

### Example 4

### Synthesis of (S)-N-[1-(cyanomethyl-carbamoyl)-2-(3-ethyl-4-hydroxy-5-iodo-phenyl)-ethyl]4-morpholin-4-yl-benzamide (compound 8)

Step 1

In a 500 mL roundbottom flask was placed p-morpholinobenzoic acid hydrochloride (2.0 g, 8.2 mmol), diiodotyrosine ethyl ester bisulfate (3.78 g, 8.2 mmol) and HATU (3.15 g, 8.29 mmol). The solids were taken up with vigorous stirring in DMF (50 mL) and triethylamine (6 mL). The reaction was allowed to proceed overnight. The reaction solution was concentrated in vacuo to give a red oil, which was eluted through a plug of silica gel using ethyl acetate. The eluted material was concentrated in vacuo to give a yellow solid. This was dissolved in a minimum amount of boiling methanol. The methanolic solution was allowed to cool to room temperature and then made to stir vigorously while the slow addition of water led to the precipitation of yellow solids. The mixture was refrigerated overnight. The precipitate was filtered and dried to give 3-(3,5-diiodo-4-hydroxy-phenyl)-2-(4-morphilin-4-yl-benzoylamino)-propionic acid ethyl ester (3.31 g) as a yellow solid.

Step 2

In a 100 mL roundbottom flask equipped with stir bar was placed methyltin trichloride (1.00 g, 4.17 mmol). The solid was taken up in 10% aqueous KOH that was sparged with nitrogen and stirred. To this solution was added a catalytic amount (5 mg) of palladium(II) chloride followed by the rapid addition of 3-(3,5-diiodo-4-hydroxy-phenyl)-2-(4-morpholin-4-yl-benzoylamino)-propionic acid ethyl ester (1.11 g, 1.71 mmol). The reaction was heated to 90° C. and stirred for 14 hours. The solution was acidified with 1.0 N HCl and extracted with ethyl acetate. The organic fraction was dried over MgSO₄. Filtration and solvent evaporation gave a crude product, which was dissolved in DMF and 1.0 mL triethylamine followed by addition of HATU (0.195 g, 0.512 mmol) and aminoacetonitrile hydrochloride (0.070 g, 0.757 mmol). After 4 hours, the reaction mixture was poured into ethyl acetate and washed with 10% citric acid, saturated sodium bicarbonate and brine. The organic fraction was dried over MgSO₄. Filtration and solvent evaporation followed by reversed-phase HPLC purification of the residue gave the (S)-N-[1-(cyanomethyl-carbamoyl)-2-(3,5-diethyl-4-hydroxy-5-iodo-phenyl)-ethyl]-4-morpholin-4-yl-benzamide as the trifluoroacetate salt (0.049 g) as a white solid (a 7% overall yield). Proton NMR (300 MHz, DMSO-d6) δ 8.7 (t, 1H) d 8.3 (t, 1H) δ 7.7 (d, 2H) δ 6.9 (m, 4H) δ 6.9 (d, 2H) δ 4.5 (m, 1H) δ 4.1 (d, 2H) δ 3.7 (m, 4H) δ 3.6 (s, 3H) δ 3.2 (m, 4H) δ 3.0-2.8 (m, 2H) δ 2.1 (s, 6H). MS (electrospray): mH⁺ 450.5 (100%) and N-[1-(cyanomethyl-carbamoyl)-2-(4-hydroxy-3-iodo-5-methylphenyl)-ethyl]-4-morpholin-4-yl-benzamide. Proton NMR (300 MHz, DMSO-d6) δ 8.6 (t, 1H) δ 8.3 (t, 1H) δ 7.7 (d, 2H) δ 7.5 (s, 1H) δ 7.0 (s, 1H) δ 6.9 (d, 2H) δ 4.5 (m, 1H) δ 4.1 (m, 2H) δ 3.7 (m, 4H) δ 3.2 (m, 4H) δ 3.0-2.8 (m, 2H) δ 2.1 (s, 3H). MS (electrospray): mH⁺ 549.2 (100%).

### Example 5

### Synthesis of (S)-N-[1-(cyanomethyl-carbamoyl)-2-(3-ethyl-4-hydroxy-5-iodo-phenyl)-ethyl]4-morpholin-4-yl-benzamide (compound 8)

Step 1

In a sealed tube equipped with a stir bar were placed 3-(4-hydroxy-3-iodophenyl)-2-(4-morpholin-4-yl-benzoylamino)-propionic acid ethyl ester (1.15 g, 2.14 mmol). The solid was taken up in 1,4-dioxane (5 mL) and triethylamine (1 mL). To the stirring solution was added dichlorobis-(triphenylphosphine)palladium (II) (15 mg) and copper(I) iodide (7 mg) followed by (trimethylsilyl)acetylene (0.34 mL). The reaction mixture was sealed and placed in a 60° C. oil bath and allowed to proceed overnight. The mixture was diluted with ethyl acetate and filtered through celite filter aid. The organic solution was washed with 1.0 N HCl, sodium bicarbonate and brine. The solution was dried over MgSO₄. Filtration and solvent evaporation gave a thick oily residue that was used in the next step without purification.

Step 2

The crude product from Step 1 above, was taken up in tetrabutylammonium fluoride (10 mL of a 1.0 M solution in THF). After one hour, the solution was again concentrated in vacuo. This residues were then taken up in ethanol with 10% palladium on carbon (25 mg) and hydrogenated in a Parr shaker overnight. After filtering through celite and concentrating in vacuo, the residue was chromatographed on silica gel using 1:1 hexane:ethyl acetate as the eluent. The crude product was then taken up in 15 mL dichloromethane and cooled to 0° C. in an ice bath. To this solution were added aluminum trichloride (0.9 g, 6.75 mmol) and ethanethiol (0.950 mL, 12.8 mmol). The mixture was allowed to warm to RT while stirring for 2 hours. LCMS indicated complete demethylation of the phenol. Volatiles were removed in vacuo. Residues were dissolved in dichloromethane and washed with 1.0 N HCl, saturated sodium bicarbonate and brine. The organic fraction was dried over MgSO₄ and filtered. Volatiles were removed in vacuo. Potassium iodide (0.115 g, 0.697 mmol) was added to the solid residues and the two were taken up in ammonium hydroxide with stirring followed by the addition of iodine crystals (0.177 g, 0.697 mmol). The solution quickly decolorized and was allowed to stir for one hour. The reaction was neutralized with the addition of acetic acid. The mixture was then transferred to a separatory funnel and extracted with dichloromethane. LCMS of the organic extract showed only the desired product.

The residues obtained from removal of solvent were taken up in 10 mL of tetrahydrofuran and 10 mL of water, followed by the addition of lithium hydroxide (0.350 g, 8.34 mmol). After stirring for one hour, the reaction mixture was acidified with the addition of acetic acid. The reaction mixture was again extracted with dichloromethane. The organic layer was dried in vacuo. The solids obtained were taken up in DMF and triethylamine with stirring, followed by the addition of HATU (0.203 g, 0.509 mmol) and aminoacetonitrile hydrochloride (0.060 g, 0.650 mmol) and allowed to stir overnight. Solvents were removed in vacuo and the residues were purified by reversed phase HPLC to give 10 mg of the title compound as a waxy white solid (a 2.5% overall yield). Proton NMR (300 MHz, DMSO-d6): δ 8.8 (t, 1H) δ 8.4 (d, 1H) δ 7.7 (d, 2H) δ 7.5 (s, 1H) δ 7.05 (s, 1H) δ 6.95 (d, 2H) δ 4.5 (m, 1H) δ 4.1 (m, 2H) δ 3.7 (m, 4H) δ 3.2 (m, 4H) δ 3.0-2.8 (m, 2H) δ 2.5 (q, 2H) δ 2.1 (t, 3H). MS (electrospray): mH⁺ 562.4 (100%).

### FORMULATION EXAMPLES

Representative Pharmaceutical Formulations Containing a Compound of Formula I are as described below:

### Example 1

### Oral Formulation

| | | |
|---|---|---|
| Compound of Formula I | 10-100 | mg |
| Citric Acid Monohydrate | 105 | mg |
| Sodium Hydroxide | 18 | mg |
| Flavoring | | |
| water | qs to 100 | ml |

### Example 2

### Intravenous Formulation

| Compound of Formula I | 0.1 to 10 | mg |
|---|---|---|
| Dextrose Monohydrate | q.s. to make isotonic | |
| Citric Acid Monohydrate | 1.05 | mg |
| Sodium Hydroxide | 0.18 | mg |
| Water for Injection | q.s. to 1.0 | mL |

### Example 3

### Tablet Formulation

| | |
|---|---|
| Compound of Formula I | 1% |
| Microcrystalline Cellulose | 73% |
| Stearic Acid | 25% |
| Colloidal Silica | 1% |

### BIOLOGICAL EXAMPLES

### Example 1

### Cathepsin B Assay

Solutions of test compounds (varying concentrations in 10 μL of DMSO) were diluted into assay buffer (40 μL, comprising: MES, 50 mM (pH 6); polyoxyethylenesorbitan monolaurate, 0.001%; EDTA (2.5 mM); and DTT, 2.5 mM). Human Cathepsin B (0.1 pMoles in 25 μL of assay buffer) was added to the dilutions. The assay solutions were mixed for 5-10 seconds on a shaker plate, covered and incubated for 30 minutes at room temperature. BOC-LKR-AMC (8 nMoles in 25 μL of assay buffer) was added to the assay solutions and hydrolysis was followed by fluorescence spectroscopy (ex 355 nm, em 460 nm) for 5 minutes. Apparent inhibition constants (Kᵢ) were calculated from the enzyme progress curves using standard mathematical models.

Compounds of the invention were tested by the above-described assay and observed to exhibit Cathepsin B inhibitory activity.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled. All patents, patent applications and publications cited in this application are hereby incorporated by reference in their entirety for all purposes to the same extent as if each individual patent, patent application or publication were so individually denoted.

1. A compound of Formula I: 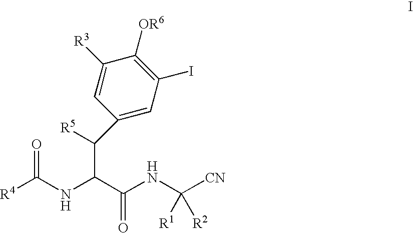
wherein:
R¹ and R² are independently hydrogen, alkyl, haloalkyl, hydroxyalkyl, aryl, or aralkyl; or
R¹ and R² together with the carbon atom to which they are attached form cycloalkyl or heterocycloalkyl;
R³ is alkyl or iodo; and
R⁴ is selected from the group consisting of aryl, heteroaryl, or heterocycloalkyl wherein R⁴ is optionally substituted with one, two or three R^{a} wherein:
each R^{a} is independently selected from the group consisting of alkyl, alkoxy, hydroxy, alkylthio wherein the sulfur may be oxidized to sulfoxide or sulfone, halo, haloalkyl, haloalkoxy, nitro, amino, alkylamino, dialkylamino, aminocarbonyl, alkylaminocarbonyl, dialkylaminocarbonyl, aminosulfonyl, alkylaminosulfonyl, dialkylaminosulfonyl, aryl, heteroaryl, heterocycloalkyl, arylamino, heteroarylamino, heterocycloalkylamino, aryloxy, heteroaryloxy, heterocycloalkyloxy, arylthio wherein the sulfur may be oxidized to sulfoxide or sulfone, heteroarylthio wherein the sulfur may be oxidized to sulfoxide or sulfone, heterocycloalkylthio wherein the sulfur may be oxidized to sulfoxide or sulfone, cyano, acyl, carboxy, or alkoxycarbonyl wherein R^{a} is optionally substituted with one, two or three R^{b} wherein:
each R^{b} is independently selected from the group consisting of alkyl, alkoxy, hydroxy, alkylthio wherein the sulfur may be oxidized to sulfoxide or sulfone, halo, haloalkyl, haloalkoxy, nitro, amino, alkylamino, dialkylamino, aminocarbonyl, alkylaminocarbonyl, dialkylaminocarbonyl, aminosulfonyl, alkylaminosulfonyl, dialkylaminosulfonyl, aryl, heteroaryl, heterocycloalkyl, arylamino, heteroarylamino, heterocycloalkylamino, aryloxy, heteroaryloxy, heterocycloalkyloxy, arylthio wherein the sulfur may be oxidized to sulfoxide or sulfone, heteroarylthio wherein the sulfur may be oxidized to sulfoxide or sulfone, heterocycloalkylthio wherein the sulfur may be oxidized to sulfoxide or sulfone, cyano, acyl, carboxy, or alkoxycarbonyl wherein each R^{b} is optionally substituted with one, two or three substituents independently selected from alkyl, alkoxy, hydroxy, alkylthio wherein the sulfur may be oxidized to sulfoxide or sulfone, halo, haloalkyl, haloalkoxy, carboxy, alkoxycarbonyl, amino, alkylamino, dialkylamino, aminocarbonyl, alkylaminocarbonyl, dialkylaminocarbonyl, aminosulfonyl, alkylaminosulfonyl, dialkylaminosulfonyl, cyano, or nitro;
R⁵ and R⁶ are independently hydrogen or alkyl;
or a pharmaceutically acceptable salt thereof.

2. The compound of claim 1 wherein R¹ and R² are hydrogen.

3. The compound of claim 1 wherein R¹ and R² form cycloalkyl.

4. The compound of claim 1 wherein R¹ and R² form heterocycloalkyl.

5. The compound of claim 1 wherein wherein R¹ is hydrogen and R² is haloalkyl.

6. The compound of any of the claims 2-6 wherein:
R⁵ is hydrogen or methyl; and R⁶ is hydrogen or methyl.

7. The compound of claim 6 wherein R³ is alkyl.

8. The compound of claim 6 wherein R³ is iodo.

9. The compound of claim 7 wherein:
R⁴ is aryl, heteroaryl, or heterocyloalkyl optionally substituted with one, two or three R^{a} wherein:
each R^{a} is independently selected from the group consisting of alkyl, alkoxy, hydroxy, alkylthio wherein the sulfur may be oxidized to sulfoxide or sulfone, halo, haloalkyl, haloalkoxy, nitro, amino, alkylamino, dialkylamino, aminocarbonyl, alkylaminocarbonyl, dialkylaminocarbonyl, aminosulfonyl, alkylaminosulfonyl, dialkylaminosulfonyl, aryl, heteroaryl, heterocycloalkyl, arylamino, heteroarylamino, heterocycloalkylamino, aryloxy, heteroaryloxy, heterocycloalkyloxy, arylthio wherein the sulfur may be oxidized to sulfoxide or sulfone, heteroarylthio wherein the sulfur may be oxidized to sulfoxide or sulfone, heterocycloalkylthio wherein the sulfur may be oxidized to sulfoxide or sulfone, cyano, acyl, carboxy, or alkoxycarbonyl wherein R^{a} is optionally substituted with one, two or three R^{b} wherein:
each R^{b} is independently selected from the group consisting of alkyl, alkoxy, hydroxy, alkylthio wherein the sulfur may be oxidized to sulfoxide or sulfone, halo, haloalkyl, haloalkoxy, nitro, amino, alkylamino, dialkylamino, aminocarbonyl, alkylaminocarbonyl, dialkylaminocarbonyl, aminosulfonyl, alkylaminosulfonyl, dialkylaminosulfonyl, cyano, acyl, carboxy, or alkoxycarbonyl.

10. The compound of claim 8 wherein:
R⁴ is aryl, heteroaryl, or heterocyloalkyl optionally substituted with one, two or three R^{a} wherein:
each R^{a} is independently selected from the group consisting of alkyl, alkoxy, hydroxy, alkylthio wherein the sulfur may be oxidized to sulfoxide or sulfone, halo, haloalkyl, haloalkoxy, nitro, amino, alkylamino, dialkylamino, aminocarbonyl, alkylaminocarbonyl, dialkylaminocarbonyl, aminosulfonyl, alkylaminosulfonyl, dialkylaminosulfonyl, aryl, heteroaryl, heterocycloalkyl, arylamino, heteroarylamino, heterocycloalkylamino, aryloxy, heteroaryloxy, heterocycloalkyloxy, arylthio wherein the sulfur may be oxidized to sulfoxide or sulfone, heteroarylthio wherein the sulfur may be oxidized to sulfoxide or sulfone, heterocycloalkylthio wherein the sulfur may be oxidized to sulfoxide or sulfone, cyano, acyl, carboxy, or alkoxycarbonyl wherein R^{a} is optionally substituted with one, two or three R^{b} wherein:
each R^{b} is independently selected from the group consisting of alkyl, alkoxy, hydroxy, alkylthio wherein the sulfur may be oxidized to sulfoxide or sulfone, halo, haloalkyl, haloalkoxy, nitro, amino, alkylamino, dialkylamino, aminocarbonyl, alkylaminocarbonyl, dialkylaminocarbonyl, aminosulfonyl, alkylaminosulfonyl, dialkylaminosulfonyl, cyano, acyl, carboxy, or alkoxycarbonyl.

11. The compound of claim 7 wherein:
R⁴ is selected from the group consisting of aryl, heteroaryl, or heterocycloalkyl wherein R⁴ is optionally substituted with one, two or three R^{a} wherein:
each R^{a} is independently selected from the group consisting of alkyl, alkoxy, hydroxy, alkylthio wherein the sulfur may be oxidized to sulfoxide or sulfone, halo, haloalkyl, haloalkoxy, nitro, amino, alkylamino, dialkylamino, aminocarbonyl, alkylaminocarbonyl, dialkylaminocarbonyl, aminosulfonyl, alkylaminosulfonyl, dialkylaminosulfonyl, aryl, heteroaryl, heterocycloalkyl, arylamino, heteroarylamino, heterocycloalkylamino, aryloxy, heteroaryloxy, heterocycloalkyloxy, arylthio wherein the sulfur may be oxidized to sulfoxide or sulfone, heteroarylthio wherein the sulfur may be oxidized to sulfoxide or sulfone, heterocycloalkylthio wherein the sulfur may be oxidized to sulfoxide or sulfone, cyano, acyl, carboxy, or alkoxycarbonyl provided that R⁴ is substituted with at least one R^{a} that is an aryl, heteroaryl or heterocycloalkyl ring or a group that has an aryl, heteroaryl or heterocyclic ring and further wherein R^{a} is optionally substituted with one, two or three R^{b} wherein:
each R^{b} is independently selected from the group consisting of alkyl, alkoxy, hydroxy, alkylthio wherein the sulfur may be oxidized to sulfoxide or sulfone, halo, haloalkyl, haloalkoxy, nitro, amino, alkylamino, dialkylamino, aminocarbonyl, alkylaminocarbonyl, dialkylaminocarbonyl, aminosulfonyl, alkylaminosulfonyl, dialkylaminosulfonyl, aryl, heteroaryl, heterocycloalkyl, arylamino, heteroarylamino, heterocycloalkylamino, aryloxy, heteroaryloxy, heterocycloalkyloxy, arylthio wherein the sulfur may be oxidized to sulfoxide or sulfone, heteroarylthio wherein the sulfur may be oxidized to sulfoxide or sulfone, heterocycloalkylthio wherein the sulfur may be oxidized to sulfoxide or sulfone, cyano, acyl, carboxy, or alkoxycarbonyl provided that R^{a} is substituted with at least one R^{b} that is an aryl, heteroaryl or heterocycloalkyl ring or a group that has an aryl, heteroaryl or heterocyclic ring wherein each R^{b} is optionally substituted with one, two or three substituents independently selected from alkyl, alkoxy, hydroxy, alkylthio wherein the sulfur may be oxidized to sulfoxide or sulfone, halo, haloalkyl, haloalkoxy, carboxy, alkoxycarbonyl, amino, alkylamino, dialkylamino, aminocarbonyl, alkylaminocarbonyl, dialkylaminocarbonyl, aminosulfonyl, alkylaminosulfonyl, dialkylaminosulfonyl, cyano, or nitro.

12. The compound of claim 8 wherein:
R⁴ is selected from the group consisting of aryl, heteroaryl, or heterocycloalkyl wherein R⁴ is optionally substituted with one, two or three R^{a} wherein:
each R^{a} is independently selected from the group consisting of alkyl, alkoxy, hydroxy, alkylthio wherein the sulfur may be oxidized to sulfoxide or sulfone, halo, haloalkyl, haloalkoxy, nitro, amino, alkylamino, dialkylamino, aminocarbonyl, alkylaminocarbonyl, dialkylaminocarbonyl, aminosulfonyl, alkylaminosulfonyl, dialkylaminosulfonyl, aryl, heteroaryl, heterocycloalkyl, arylamino, heteroarylamino, heterocycloalkylamino, aryloxy, heteroaryloxy, heterocycloalkyloxy, arylthio wherein the sulfur may be oxidized to sulfoxide or sulfone, heteroarylthio wherein the sulfur may be oxidized to sulfoxide or sulfone, heterocycloalkylthio wherein the sulfur may be oxidized to sulfoxide or sulfone, cyano, acyl, carboxy, or alkoxycarbonyl provided that R⁴ is substituted with at least one R^{a} that is an aryl, heteroaryl or heterocycloalkyl ring or a group that has an aryl, heteroaryl or heterocyclic ring and further wherein R^{a} is optionally substituted with one, two or three R^{b} wherein:
each R^{b} is independently selected from the group consisting of alkyl, alkoxy, hydroxy, alkylthio wherein the sulfur may be oxidized to sulfoxide or sulfone, halo, haloalkyl, haloalkoxy, nitro, amino, alkylamino, dialkylamino, aminocarbonyl, alkylaminocarbonyl, dialkylaminocarbonyl, aminosulfonyl, alkylaminosulfonyl, dialkylaminosulfonyl, aryl, heteroaryl, heterocycloalkyl, arylamino, heteroarylamino, heterocycloalkylamino, aryloxy, heteroaryloxy, heterocycloalkyloxy, arylthio wherein the sulfur may be oxidized to sulfoxide or sulfone, heteroarylthio wherein the sulfur may be oxidized to sulfoxide or sulfone, heterocycloalkylthio wherein the sulfur may be oxidized to sulfoxide or sulfone, cyano, acyl, carboxy, or alkoxycarbonyl provided that R^{a} is substituted with at least one R^{b} that is an aryl, heteroaryl or heterocycloalkyl ring or a group that has an aryl, heteroaryl or heterocyclic ring wherein each R^{b} is optionally substituted with one, two or three substituents independently selected from alkyl, alkoxy, hydroxy, alkylthio wherein the sulfur may be oxidized to sulfoxide or sulfone, halo, haloalkyl, haloalkoxy, carboxy, alkoxycarbonyl, amino, alkylamino, dialkylamino, aminocarbonyl, alkylaminocarbonyl, dialkylaminocarbonyl, aminosulfonyl, alkylaminosulfonyl, dialkylaminosulfonyl, cyano, or nitro.

13. The compound of claim 1 wherein R¹ and R² are hydrogen; and
R⁴ is selected from the group consisting of aryl, heteroaryl, or heterocycloalkyl wherein R⁴ is optionally substituted with one, two or three R^{a} wherein:
each R^{a} is independently selected from the group consisting of alkyl, alkoxy, hydroxy, alkylthio wherein the sulfur may be oxidized to sulfoxide or sulfone, halo, haloalkyl, haloalkoxy, nitro, amino, alkylamino, dialkylamino, aminocarbonyl, alkylaminocarbonyl, dialkylaminocarbonyl, aminosulfonyl, alkylaminosulfonyl, dialkylaminosulfonyl, aryl, heteroaryl, heterocycloalkyl, arylamino, heteroarylamino, heterocycloalkylamino, aryloxy, heteroaryloxy, heterocycloalkyloxy, arylthio wherein the sulfur may be oxidized to sulfoxide or sulfone, heteroarylthio wherein the sulfur may be oxidized to sulfoxide or sulfone, heterocycloalkylthio wherein the sulfur may be oxidized to sulfoxide or sulfone, cyano, acyl, carboxy, or alkoxycarbonyl provided that R⁴ is substituted with at least one R^{a} that is an aryl, heteroaryl or heterocycloalkyl ring or a group that has an aryl, heteroaryl or heterocyclic ring and further wherein R^{a} is optionally substituted with one, two or three R^{b} wherein:
each R^{b} is independently selected from the group consisting of alkyl, alkoxy, hydroxy, alkylthio wherein the sulfur may be oxidized to sulfoxide or sulfone, halo, haloalkyl, haloalkoxy, nitro, amino, alkylamino, dialkylamino, aminocarbonyl, alkylaminocarbonyl, dialkylaminocarbonyl, aminosulfonyl, alkylaminosulfonyl, dialkylaminosulfonyl, aryl, heteroaryl, heterocycloalkyl, arylamino, heteroarylamino, heterocycloalkylamino, aryloxy, heteroaryloxy, heterocycloalkyloxy, arylthio wherein the sulfur may be oxidized to sulfoxide or sulfone, heteroarylthio wherein the sulfur may be oxidized to sulfoxide or sulfone, heterocycloalkylthio wherein the sulfur may be oxidized to sulfoxide or sulfone, cyano, acyl, carboxy, or alkoxycarbonyl provided that R^{a} is substituted with at least one R^{b} that is an aryl, heteroaryl or heterocycloalkyl ring or a group that has an aryl, heteroaryl or heterocyclic ring wherein each R^{b} is optionally substituted with one, two or three substituents independently selected from alkyl, alkoxy, hydroxy, alkylthio wherein the sulfur may be oxidized to sulfoxide or sulfone, halo, haloalkyl, haloalkoxy, carboxy, alkoxycarbonyl, amino, alkylamino, dialkylamino, aminocarbonyl, alkylaminocarbonyl, dialkylaminocarbonyl, aminosulfonyl, alkylaminosulfonyl, dialkylaminosulfonyl, cyano, or nitro.

14. A pharmaceutical composition comprising a compound of any of the claim 1-14 in admixture with one or more pharmaceutically suitable excipients.

15. A method of treating a disease in an animal in which inhibition of Cathepsin B, can prevent, inhibit or ameliorate the pathology and/or symptomatology of the disease, which method comprises administering to the animal a pharmaceutical composition comprising a therapeutically effective amount of compound of any of the claims 1-14 or a pharmaceutically acceptable salt thereof.
